(19) United States
(12) Patent Application Publication
(10) Pub. No.: US 2009/0216089 A1
(43) Pub. Date: Aug. 27, 2009

(54) FOOT RETRACTORS

(75) Inventor: Richard S. Davidson, Haverford, PA (US)

Correspondence Address: RATNERPRESTIA, P.O. BOX 980, VALLEY FORGE, PA, 19482, US

(73) Assignee: THE CHILDREN'S HOSPITAL OF PHILADELPHIA, PHILADELPHIA, PA (US)

(21) Appl. No.: 12/094,557

(22) PCT Filed: Nov. 21, 2006
(86) PCT No.: PCT/US06/45041

§ 371 Date: Dec. 22, 2008

Related U.S. Application Data
(60) Provisional application No. 60/738,627, filed on Nov. 21, 2005.

(51) Int. Cl. A61B 1/32

(52) U.S. Cl. 600/235

(57) ABSTRACT

A foot retractor includes an elongated body portion, a handle curving away from the body portion, and a tapered blade portion extending at an angle from the body portion, distal from the handle portion. The blade portion comprises a curvature therein.

### CROSS REFERENCE TO RELATED APPLICATIONS

The present application claims priority from U.S. Provisional Patent Application Ser. No. 60/738,627, filed on Nov. 21, 2005.

### FIELD OF THE INVENTION

The present invention relates to foot retractors that are designed specifically to optimize retraction of soft tissues about the bones of the foot, while maintaining the assistant's hands comfortably outside the operating field.

### BACKGROUND OF THE INVENTION

The anatomy of the foot is unique within the human body. As in the hand, there is a complex inter-relationship between bones, ligaments, tendons, and vessels. Unlike the hand, however, the foot is wider and, while triangular posteriorly, becomes flat toward the forefoot. Corrective osteotomy in the foot often requires that retractors fit around the tarsal bones to protect soft tissues on the side opposite the incision (the blind side). Retractors, however, need to fit between bones into small spaces such as the sinus tarsi or the posterior ankle, and under the neck of the talus. Existing devices do not hook around bones, are prone to slipping out of position, and create operating fields that are awkward to work in, such as are found in the foot. It would be beneficial to develop retractors that may be used specifically in the foot.

### SUMMARY OF THE INVENTION

Briefly, the present invention provides a foot retractor. In accordance with one aspect of the invention, a foot retractor includes an elongated body portion, a handle curving away from the body portion, and a tapered blade portion extending at an angle from the body portion, distal from the handle portion. The blade portion comprises a curvature therein.

In accordance with another aspect of the invention, a foot retractor includes a generally elongated body portion extending between a proximal end and a distal end and a handle extending from the proximal end. The handle extends on one side of the body portion. A blade portion extends from the distal end. The blade portion further comprises a blade having a curvature therein and a blade tip extending distally from the blade. The retractor tapers from the handle to the blade tip.

In accordance with another aspect of the invention, a foot retractor includes a tapered body portion extending in a plane, a handle extending away from the tapered body portion on a first side of the plane, and a tapered blade portion extending away from the body portion, distal from the handle, on the first side of the plane.

The present invention also provides kits comprising a plurality of retractors according to the present invention.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain features of the invention. In the drawings:

FIG. 1 is a perspective view of a foot retractor according to a first embodiment of the present invention;

FIG. 2 is a side elevation view of the foot retractor of FIG. 1;

FIG. 3 is a top plan view of the foot retractor of FIG. 1;

FIG. 4 is a distal elevation view of the foot retractor of FIG. 1;

FIG. 5 is a perspective view of a foot retractor according to a second embodiment of the present invention;

FIG. 6 is a side elevation view of the foot retractor of FIG. 5;

FIG. 7 is a top plan view of the foot retractor of FIG. 5;

FIG. 8 is a distal elevation view of the foot retractor of FIG. 5;

FIG. 9 is a perspective view of a foot retractor according to a third embodiment of the present invention;

FIG. 10 is a side elevation view of the foot retractor of FIG. 9;

FIG. 11 is a top plan view of the foot retractor of FIG. 9;

FIG. 12 is a distal elevation view of the foot retractor of FIG. 9;

FIG. 13 is a perspective view of a foot retractor according to a fourth embodiment of the present invention;

FIG. 14 is a side elevation view of the foot retractor of FIG. 13;

FIG. 15 is a top plan view of the foot retractor of FIG. 13;

FIG. 16 is a distal elevation view of the foot retractor of FIG. 13;

FIG. 17 is a side elevation view of a kit comprising foot retractors shown in each of FIGS. 1, 5, 9, and 13;

FIG. 18 is a perspective view of retractors according to the present invention prior to a first exemplary application;

FIG. 19 is a perspective view of the retractors of FIG. 18 being used in the first exemplary application;

FIG. 20 is a perspective view of retractors according to the present invention prior to a second exemplary application;

FIG. 21 is a perspective view of the retractors of FIG. 20 being used in the second exemplary application; and

FIG. 22 is a perspective view of retractors according to the present invention being used in a third exemplary application.

### DETAILED DESCRIPTION OF THE INVENTION

Certain terminology is used in the following description for convenience only and is not limiting. As used herein, the term “distal” refers to a section or area of the device positioned toward the patient during use, and the term “proximal” refers to a section or area on the device positioned toward the user of the device during use. The terminology includes the words above specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Foot retractors according to the present invention are designed specifically to optimize retraction of the soft tissues around the unique shapes of the bones of the foot while maintaining the assistant's hands comfortably outside of the operating field. The present invention provides different sizes and shapes of retractors to fit the unique shape and contours of the human foot and to accommodate the varied locations between bones and small spaces in the foot for both infant and adult sized feet. For example, foot surgeries, such as corrective osteotomy, often require that retractors fit around the tarsal bones to protect soft tissues on the side opposite an incision. The retractors must fit into small spaces not found in other anatomic locations, such as the sinus tarsi, the anterior tibialis tendon, and under the neck of the talis.

Different sizes of retractors of the present invention may be used for different sized patients. For example, a foot retractor for use with an infant or small child may be significantly smaller than a similarly shaped foot retractor used on an adult. The dimensions described in this specification are for illustration only, and represent exemplary dimensions. Other dimensions may be suitable for purposes of practicing the invention.

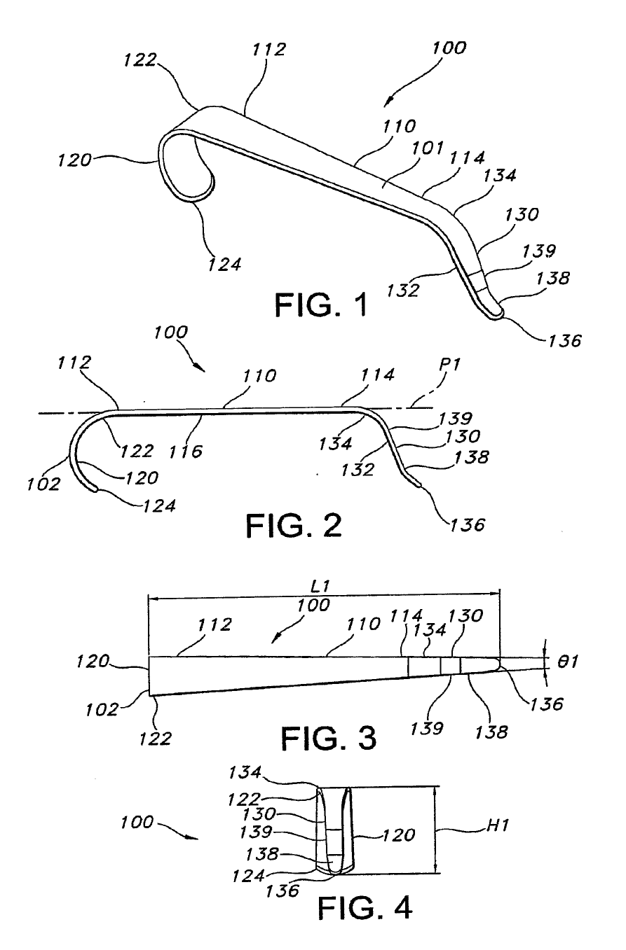

A foot retractor 100 according to a first embodiment of the present invention is shown in FIGS. 1-4. Foot retractor 100 is of unitary construction and includes a generally elongated body portion 110 extending in a plane “P1” between a proximal end 112 and a distal end 114. A handle 120 extends from proximal end 112. Handle 120 extends on one side 116 of plane P1. A blade portion 130 extends from distal end 114. Blade portion 130 extends on the one side 116 of plane P1.

In an exemplary embodiment, body 110 is approximately 2 millimeters thick. Proximal end 112 is approximately 12 millimeters wide and may taper to distal end 114, which is approximately 6 millimeters wide. Handle 120 includes an attached end 122 that curves from proximal end 112 of body 110 in a curve having a radius of curvature of approximately 15 millimeters. Handle 120 terminates at a free end 124.

Blade portion 130 includes a blade 132 having an attached end 134 that curves from distal end 114 of body and a blade tip 136. Blade portion 130 also includes a curvature 138 therein between attached end 134 and blade tip 136. Curvature 138 has a radius of curvature of between approximately 10 and 15 millimeters. Attached end 134 has a radius of curvature of approximately 13 millimeters. Blade portion 130 includes a straight portion 139 extending between attached end 134 and curvature 138. Straight portion 139 extends between approximately 12 and 23 millimeters at an angle of approximately 77 degrees from plane P1. Straight portion 139 also has a thickness of approximately 1.5 millimeters. Blade tip 136 tapers at an angle θ1 of between approximately 4 and 9 degrees.

Retractor 100 includes a top face 101 that changes in contour along the length of retractor 100. In the area of blade 130, top face 101 transitions from a generally flat surface adjacent body portion 110 to a convex curvature at attached end 134. Top face 101 returns to a generally flat contour at straight portion 139, and then transitions to a concave curvature at curvature 138. Top face 101 transitions to a generally flat contour at blade tip 136. Curvature 138 provides an approximate 34 degree bend between straight portion 139 and blade tip 136.

Referring to FIG. 3, the top plan view of retractor 100 has a Length L1 of approximately 130 millimeters. Referring to FIG. 4, retractor 100 has a height H1 of between approximately 34 and 41 millimeters.

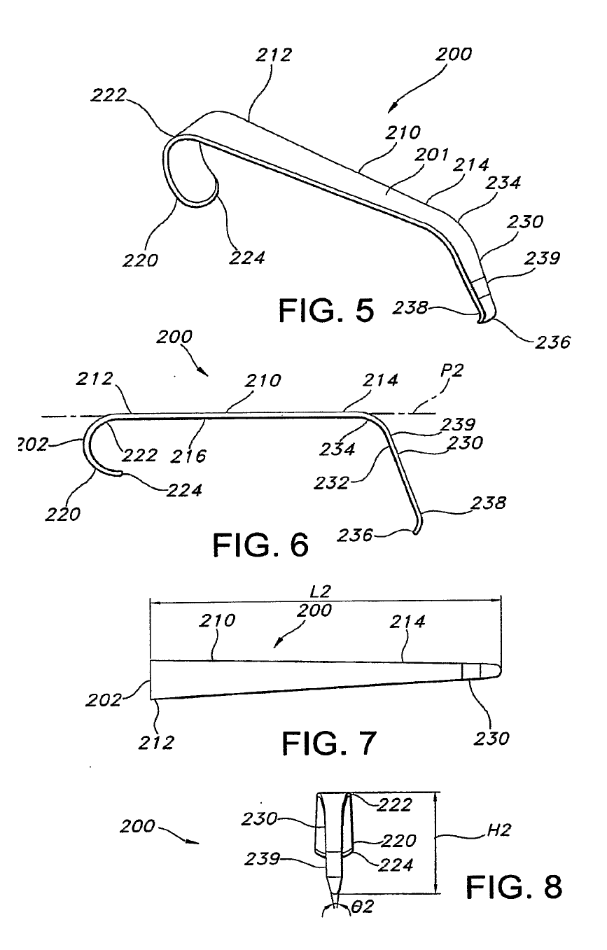

A foot retractor 200 according to a second embodiment of the present invention is shown in FIGS. 5-8. Foot retractor 200 is of unitary construction and includes a generally elongated body portion 210 extending in a plane “P2” between a proximal end 212 and a distal end 214. A handle 220 extends from proximal end 212. Handle 220 extends on one side 216 of plane P2. A blade portion 230 extends from distal end 214. Blade portion 230 extends on the one side 216 of plane P2.

In an exemplary embodiment, body 210 is approximately 2 millimeters thick. Proximal end 212 is approximately 12 millimeters wide and may taper to distal end 214, which is approximately 6 millimeters wide. Handle 220 includes an attached end 222 that curves from proximal end 212 of body 210 in a curve having a radius of curvature of approximately 8 millimeters. Handle 220 terminates at a free end 224.

Blade portion 230 includes a blade 232 having an attached end 234 that curves from distal end 214 of body and a blade tip 236. Blade portion 230 also includes a curvature 238 therein between attached end 234 and blade tip 236. Curvature 238 has a radius of curvature of between approximately 5 and 20 millimeters. Attached end 234 has a radius of curvature of approximately 6 millimeters. Blade portion 230 includes a straight portion 239 extending between attached end 234 and curvature 238. Straight portion 239 extends for approximately 23 and 32 millimeters at an angle of approximately 75 degrees from plane P2. Straight portion 239 also has a thickness of approximately 1.5 millimeters. Blade tip 236 tapers at an angle θ2 of between approximately 5 and 8 degrees.

Retractor 200 includes a top face 201 that changes in contour along the length of retractor 200. In the area of blade 230, top face 201 transitions from a generally flat surface adjacent body portion 210 to a convex curvature at attached end 234. Top face 201 returns to a generally flat contour at straight portion 239, and then transitions to a second convex curvature at curvature 238. Curvature 238 provides an approximate 19 degree bend between straight portion 239 and blade tip 236.

Referring to FIG. 7, the top plan view of retractor 200 has a length L2 of approximately 114 millimeters. Referring to FIG. 8, retractor 200 has a height H2 of between approximately 35 and 45 millimeters.

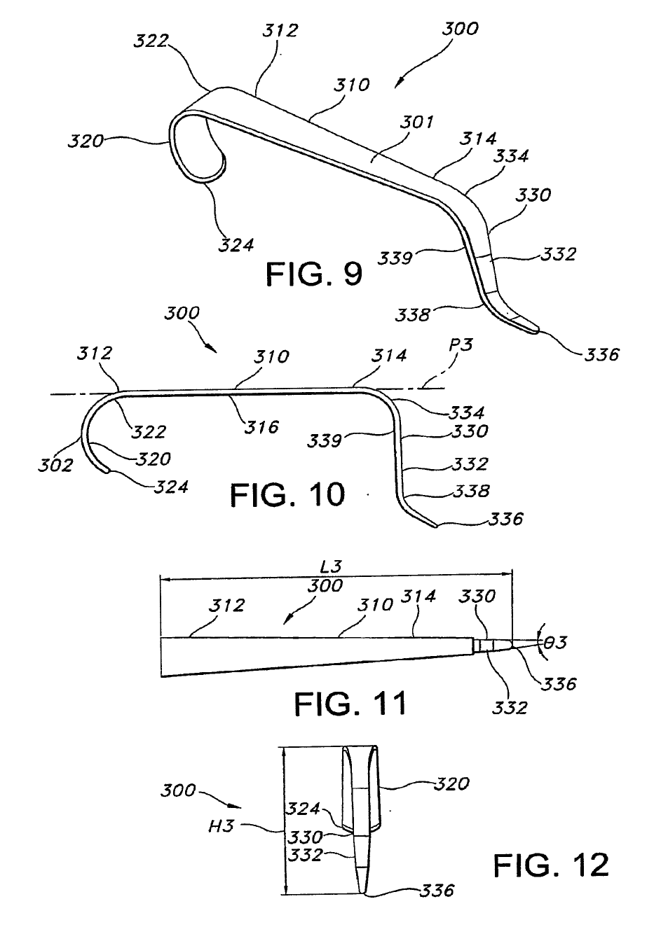

A foot retractor 300 according to a third embodiment of the present invention is shown in FIGS. 9-12. Foot retractor 300 is of unitary construction and includes a generally elongated body portion 310 extending in a plane “P3” between a proximal end 312 and a distal end 314. A handle 320 extends from proximal end 312. Handle 320 extends on one side 316 of plane P3. A blade portion 330 extends from distal end 314. Blade portion 330 extends on the one side 316 of plane P3.

In an exemplary embodiment, body 310 is approximately 2 millimeters thick. Proximal end 312 is approximately 15 millimeters wide and may taper to distal end 314, which is approximately 6 millimeters wide. Handle 320 includes an attached end 322 that curves from proximal end 312 of body 310 in a curve having a radius of curvature of approximately 13 millimeters. Handle 320 terminates at a free end 324.

Blade portion 330 includes a blade 332 having an attached end 334 that curves from distal end 314 of body and a blade tip 336. Blade portion 330 also includes a curvature 338 therein between attached end 334 and blade tip 336. Curvature 338 has a radius of curvature of between approximately 20 and 25 millimeters. Attached end 334 has a radius of curvature of approximately 11 millimeters. Blade portion 330 includes a straight portion 339 extending between attached end 334 and curvature 338. Straight portion 339 extends for between approximately 20 and 28 millimeters at an angle of approximately 90 degrees from plane P3. Straight portion 339 has a thickness of approximately 1.5 millimeters. Blade tip 336 tapers at an angle θ3 of approximately 6 degrees.

Retractor 300 includes a top face 301 that changes in contour along the length of retractor 300. In the area of blade 330, top face 301 transitions from a generally flat surface adjacent body portion 310 to a convex curvature at attached end 334. Top face 301 returns to a generally flat contour at straight portion 339, and then transitions to a concave curvature at curvature 338. Top face 301 transitions to a generally flat contour at blade tip 336. Curvature 338 provides an approximate 50 degree bend between straight portion 339 and blade tip 336.

Referring to FIG. 11, the top plan view of retractor 300 has a length L3 of approximately 130 millimeters. Referring to FIG. 12, retractor 300 has a height H3 of between approximately 51 and 62 millimeters.

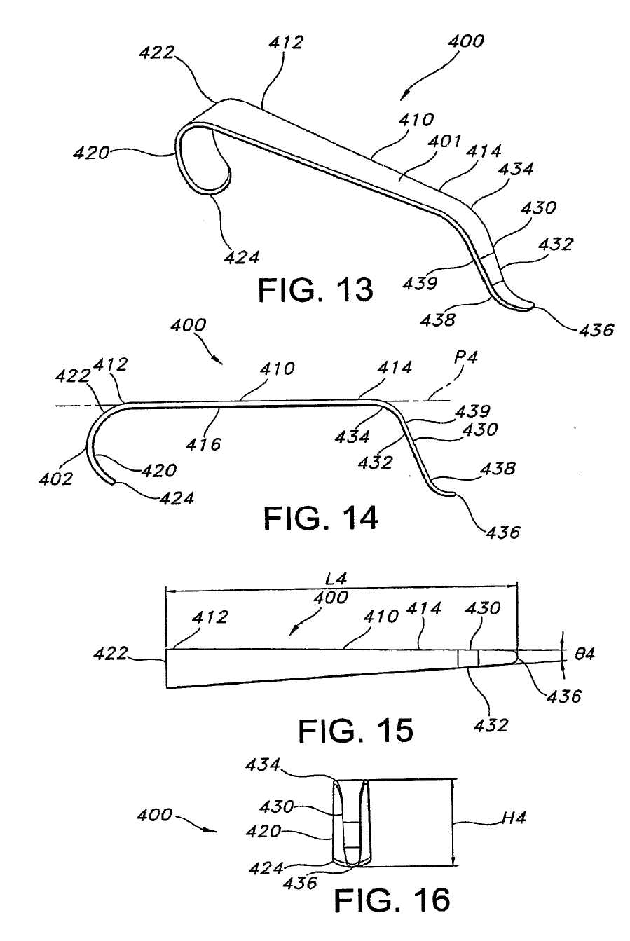

A foot retractor 400 according to a fourth embodiment of the present invention is shown in FIGS. 13-16. Foot retractor 400 is of unitary construction and includes a generally elongated body portion 410 extending in a plane “P4” between a proximal end 412 and a distal end 414. A handle 420 extends from proximal end 412. Handle 420 extends on one side 416 of plane P4. A blade portion 430 extends from distal end 414. Blade portion 430 extends on the one side 416 of plane P4.

In an exemplary embodiment, body 410 is approximately 2 millimeters thick. Proximal end 412 is approximately 12 millimeters wide and may taper to distal end 414, which is approximately 6 millimeters wide. Handle 420 includes an attached end 422 that curves from proximal end 412 of body 410 in a curve having a radius of curvature of approximately 15 millimeters. Handle 420 terminates at a free end 424.

Blade portion 430 includes a blade 432 having an attached end 434 that curves from distal end 414 of body and a blade tip 436. Blade portion 430 also includes a curvature 438 therein between attached end 434 and blade tip 436. Curvature 438 has a radius of curvature of between approximately 10 and 15 millimeters. Attached end 434 has a radius of curvature of approximately 9 millimeters. Blade portion 430 includes a straight portion 439 extending between attached end 434 and curvature 438. Straight portion 439 extends for between approximately 20 and 24 millimeters at an angle of approximately 70 degrees from plane P4. Straight portion 439 also has a thickness of approximately 1.5 millimeters. Blade tip 436 tapers at an angle θ4 of between approximately 4 and 9 degrees.

Retractor 400 includes a top face 401 that changes in contour along the length of retractor 400. In the area of blade 430, top face 401 transitions from a generally flat surface adjacent body portion 410 to a convex curvature at attached end 434. Top face 401 returns to a generally flat contour at straight portion 439, and then transitions to a concave curvature at curvature 438. Top face 401 transitions to a generally flat contour at blade tip 436. Curvature 438 provides an approximate 40 degree bend between straight portion 439 and blade tip 436.

Referring to FIG. 15, the top plan view of retractor 400 has a length L4 of approximately 133 millimeters. Referring to FIG. 16, retractor 400 has a height H4 of between approximately 33 and 35 millimeters.

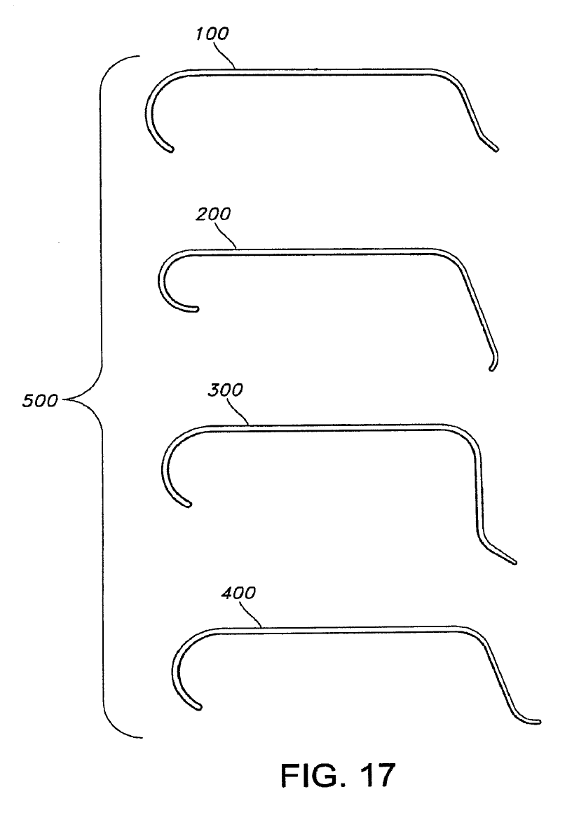

Exemplary retractors 100, 200, 300, 400 are all constructed from a biocompatible material, such as surgical stainless steel, and may be part of a kit, such as kit 500, shown in FIG. 17, in which all four of retractors 100, 200, 300, 400 are packaged. Alternatively, kit 500 may feature only some of retractors 100, 200, 300, 400. Still alternatively, kit 500 may include two or more of each retractor embodiment shown in FIG. 17.

Retractors 100, 200, 300, 400 and/or kit 500 may be used to retract bones, tendons, muscles, nerves, and vessels of the foot during surgery. The narrow profile of the retractors minimizes visual obstruction of the surgeon by allowing the user's hands to remain outside of the operating field. The thin shape and weight of each retractor allows the retractor to stay in place in a patient's foot if the user releases the retractor.

The forward/concave curvatures 138, 338, 438 hook each respective retractor 100, 300, 400 under/around bones, while the backward/convex curvature 238 of retractor 200 retracts soft tissue, and protects vessels, tissues, and nerves. Such features benefit the surgeon by allowing the surgeon to gently, yet effectively access the surgical site and also benefit the patient by minimizing trauma to the patient, thereby reducing pain and recovery time for the patient.

The retractors are designed to expose bones and joints of the foot but can, in addition, go between and around the bones of the foot, permitting isolation and exposure of individual tarsal and metatarsal bones for corrective osteotomy, and protection of the soft tissues (arteries, veins, nerves, muscles, etc) on the deep side of the foot bones from damage from sharp saws, osteotomes and displaced bone fragments.

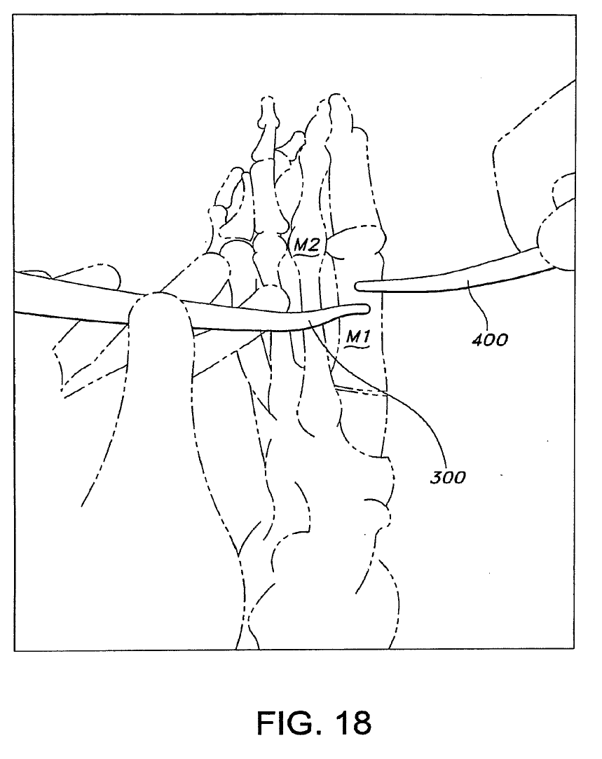

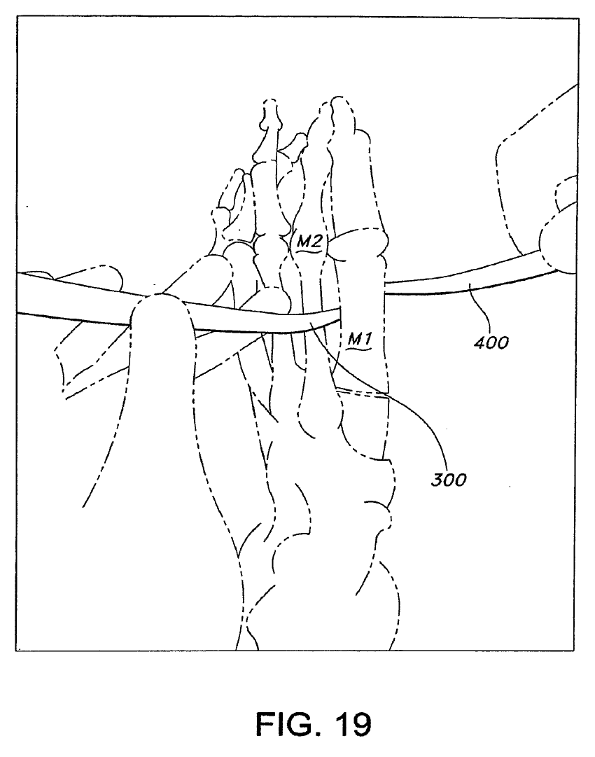

By way of example, in a procedure of retracting the first metatarsal M1, as shown in FIGS. 18 and 19, any of retractors 100, 300, 400 may be used. The selection of which of retractors 100, 300, 400 to use may depend on the anatomy of the particular foot. As shown in FIG. 19, retractors 300, 400 are placed on either side of metatarsal M1, with respective blade tips 336, 436 worked under metatarsal M1. Blade portion 330 engages second metatarsal M2 proximate to attached end 334. Second metatarsal M2 is used as a fulcrum to assist in retracting first metatarsal M1.

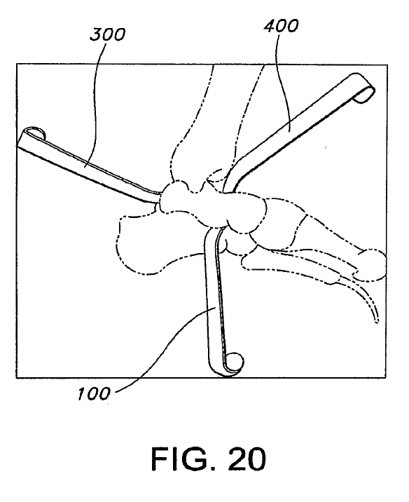

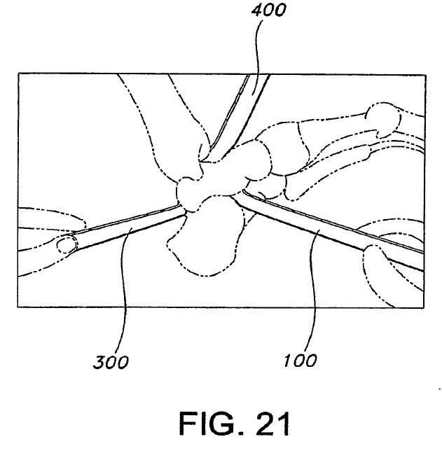

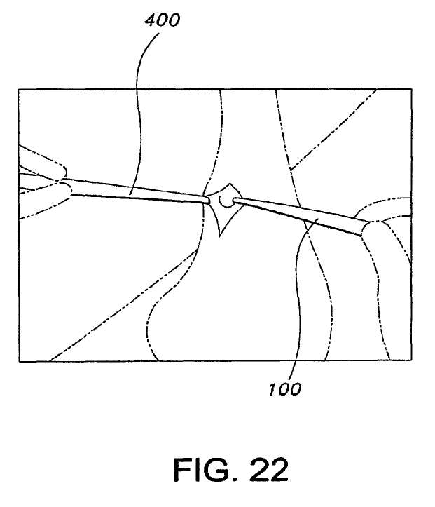

Other applications of retractors according to the present invention are shown in FIGS. 20-22. In procedures, such as medially exposing the foot, retractor 100 may be placed over the dorsum of the talar neck, head, or navicular, but under the anterior tibialis tendon and the dorsalis pedis artery. Retractor 200 may be used to retract all four layers of plantar-medial muscles, nerves, and veins to allow exposure of the mid tarsal bones and joints medially. Medial exposure of the subtalar joint may be aided by placement of retractor 300 over the dorsum of the talar neck, retracting and protecting the posterior tibialis tendon, the dorsalis pedis neurovascular bundle, and the anterior tibialis tendon. An additional retractor 100 may be placed under the neck of the talus between the talar neck and the calcaneus, retracting and protecting the abductor hallicus origin, medial muscles, medial plantar nerve, and vessels. Retractor 400 may be placed around the posterior ankle and subtalar joints, retracting and protecting the flexor digitorum longus, the flexor hallucis, and the posterior tibial neurovascular bundle.

Applications for this type of procedure include, for example, treatment of club foot, medial facet talocalcaneal coalition, accessory naviculars, infections of the midtarsal bones, osteotomy of the midtarsal bones and proximal first metatarsal, exposure of the plantar fascia, exposure of the posteromedial ankle and subtalar joints, repair of the soft tissues (tendons, nerves, and vessels of the midfoot), and fractures of the talus, sustentaculum talus, navicular and other bones of the midfoot.

By way of another example, in a procedure of laterally exposing the foot, retractor 100 is placed over the dorsum of the midfoot, but under the extensor brevis muscle, under the dorsal pedis neurovascular bundle and over the talar neck, exposing the sinus tarsi. The lateral aspect of the calcaneus is accomplished by placing retractor 100 subperiostally, around the plantar aspect of the calcaneus retracting the peroneus brevis and longus tendons. Retractor 400 is placed subperiostally, around the dorsal aspect of the calcaneus. Retractors 100, 400 may be placed all-the-way around a calcaneo-navicular coalition, protecting the soft tissues medially, as well as preventing medial displacement of bone fragments which can lead to recurrence, pain, and soft tissue damage. Retractors 100, 300, 400 may be placed around the posterior ankle, exposing the posterolateral subtalar and ankle joints.

Applications for this type of procedure include, for example, treatment of calceneus osteotomy (flatfoot, cavus, etc.), excision of calcaneo-navicular coalitions, excision of os trigone, fractures of the calcaneus, cuboid, and base of the fifth metatarsal.

While exemplary uses for each of retractors 100, 200, 300, 400 are discussed above, those skilled in the art will recognize that retractors 100, 200, 300, 400 may be used for other purposes, and on other parts of the body besides the foot.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A foot retractor comprising:
an elongated body portion;
a handle curving away from the body portion; and
a tapered blade portion extending at an angle from the body portion, distal from the handle portion, wherein the blade portion comprises a curvature therein.

2. The foot retractor according to claim 1, wherein the blade portion curves away from the body portion on the same side of the body portion as the handle.

3. The foot retractor according to claim 1, wherein the blade portion comprises an inflection point between the distal end of the body and the curvature.

4. The foot retractor according to claim 3, wherein the blade portion is adapted to curve around bone.

5. The foot retractor according to claim 1, wherein the blade portion curves away from the body portion in a first direction and the curvature of the blade also extends in the first direction.

6. The foot retractor according to claim 5, wherein the blade portion is adapted to retract soft tissue.

7. The foot retractor according to claim 1, wherein the foot retractor comprises a unitary construction.

8. The foot retractor according to claim 1, wherein the blade portion extends at an angle of less than ninety degrees with respect to the body portion.

9. A foot retractor comprising:
a generally elongated body portion extending between a proximal end and a distal end;
a handle extending from the proximal end, wherein the handle extends on one side of the body portion; and
a blade portion extending from the distal end, wherein the blade portion further comprises a blade having a curvature therein and a blade tip extending distally from the blade,
wherein the retractor tapers from the handle to the blade tip.

10. The foot retractor according to claim 9, wherein the blade portion extends on the one side of the body.

11. The foot retractor according to claim 9, wherein the blade portion comprises an inflection point between the distal end of the body and the curvature.

12. The foot retractor according to claim 11, wherein the blade portion is adapted to curve around bone.

13. The foot retractor according to claim 9, wherein the blade portion curves away from the body portion in a first direction and the curvature of the blade also extends in the first direction.

14. The foot retractor according to claim 13, wherein the blade portion is adapted to retract soft tissue.

15. The foot retractor according to claim 9, wherein the foot retractor comprises a unitary construction.

16. The foot retractor according to claim 9, wherein the blade portion extends at an angle of less than ninety degrees with respect to the body portion.

17. A foot retractor kit comprising:
at least one foot retractor according to claim 11; and
at least one foot retractor according to claim 13.

18. A foot retractor comprising:
a tapered body portion extending in a plane;
a handle extending away from the tapered body portion on a first side of the plane; and
a tapered blade portion extending away from the body portion, distal from the handle, on the first side of the plane.

19. The foot retractor according to claim 18, wherein the body portion has a first thickness and the blade portion has a second thickness, less than the first thickness.

20. The foot retractor according to claim 18, wherein the blade portion comprises a curved blade.

21. The foot retractor according to claim 18, wherein the blade portion comprises a proximal curve extending from the body portion in a first direction and a distal curve.

22. The foot retractor according to claim 21, wherein the distal curve curves in the first direction.

23. The foot retractor according to claim 21, wherein the distal curve curves in a second direction.

24. The foot retractor according to claim 18, wherein the handle comprises a curved handle portion.

25. A foot retractor kit comprising:
at least one foot retractor according to claim 22; and
at least one foot retractor according to claim 23.
